(19) United States
(12) Patent Application Publication
(10) Pub. No.: US 2025/0090538 A1
(43) Pub. Date: Mar. 20, 2025

(54) TLR7/8-ANTAGONIST FOR TREATING SJÖGREN'S SYNDROME OR MIXED CONNECTIVE TISSUE DISEASE

(71) Applicant: Novartis AG, Basel (CH)

(72) Inventors: Alexandre AVRAMEAS, Kembs (FR); Roland FEIFEL, Wallbach (CH); Stuart HAWTIN, Franken (FR); Tobias JUNT, Füllinsdorf (CH); Tamas David SHISHA, Binningen (CH)

(21) Appl. No.: 18/727,136

(22) PCT Filed: Jan. 9, 2023
(86) PCT No.: PCT/IB2023/050164

§ 371 Date: Jul. 8, 2024

Related U.S. Application Data
(60) Provisional application No. 63/298,478, filed on Jan. 11, 2022.

(51) Int. Cl. A61K 31/5377

(52) U.S. Cl. CPC A61K 31/5377

(57) ABSTRACT

The present disclosure relates to methods for treating Sjögren's Syndrome or mixed connective tissue disease using a compound of Formula (I) or a pharmaceutically acceptable salt thereof. Also disclosed herein is a compound of Formula (I) or a pharmaceutically acceptable salt thereof, for treating Sjögren's Syndrome or mixed connective tissue disease patients, as well as medicaments, dosing regimens, pharmaceutical formulations, dosage forms, and kits for use in the disclosed uses and methods.

### TECHNICAL FIELD

The present disclosure relates to methods for treating Sjögren's Syndrome (SjS) or mixed connective tissue disease (MCTD) using a TLR7 and TLR8 antagonist.

### BACKGROUND OF THE DISCLOSURE

Sjögren's syndrome (SjS) is a systemic autoimmune disease of unknown etiology characterized by lymphoid infiltration and progressive destruction of exocrine glands (Brito-Zerón P., et al, (2016) Treating the Underlying Pathophysiology of Primary Sjögren Syndrome: Recent Advances and Future Prospects. Drugs p. 1601-1623). Although the disease primarily affects the lacrimal and salivary glands, the inflammatory process can target any organ with approximately 15% of patients showing severe extraglandular manifestations (Baldini C., et al (2014) Primary Sjögren's syndrome as a multi-organ disease: impact of the serological profile on the clinical presentation of the disease in a large cohort of Italian patients. Rheumatology (Oxford) p. 839-44). The clinical presentation is most often primarily characterized by exocrinopathy of salivary and lacrimal glands presenting with dryness of the mouth and eyes. The mechanism underlying the development of SjS is the destruction of the epithelium of the exocrine glands, as a consequence of autoreactive B cells and T cells (Brito-Zerón P., et al, (2016) Treating the Underlying Pathophysiology of Primary Sjögren Syndrome: Recent Advances and Future Prospects. Drugs p. 1601-1623). The high prevalence of autoantibodies, especially against Ro/SSA, even at a very early stage of the disease suggests that autoreactive B cells participate in the pathomechanism of SjS (Nocturne G., et al, (2018) B cells in the pathogenesis of primary Sjögren syndrome. Nat Rev Rheumatol p. 133-145). Sjögren's syndrome is also linked with increased risk for malignancy, with a 10-fold elevation of lifetime-risk for B-cell lymphomas in SjS patients (Baldini et al 2014). SjS is observed in 0.3 to 1 per 1,000 individuals and its prevalence is second only to rheumatoid arthritis among systemic autoimmune disease. The disease affects mainly women with a female/male ratio of 9:1 and can occur at any age (Qin B, Wang J, Yang Z, et al (2015) Epidemiology of primary Sjögren's syndrome: a systematic review and meta-analysis. Ann. Rheum. Dis. p. 1983-9). SjS has a severe impact on quality of life and productivity, often caused by disabling fatigue associated with the disease (Mariette X., et al. (2018) Primary Sjögren's Syndrome. N. Engl. J. Med. p. 931-939).

Mixed connective tissue disease (MCTD) was first described in 1972 as a disease syndrome with overlapping features of other systemic autoimmune diseases, associated with antibodies to RNAse sensitive extractable nuclear antigen (ENA) (Sharp, et al (1972) Mixed connective tissue disease—an apparently distinct rheumatic disease syndrome associated with a specific antibody to an extractable nuclear antigen (ENA). The American journal of medicine, 52(2), pp. 148-159). Patients with MCTD often have symptoms present in SjS; notably, 42% of patients with MCTD are reported to have sicca features (Ramos-Casals et al. (2007) The overlap of Sjögren's syndrome with other systemic autoimmune diseases. Seminars in arthritis and rheumatism, 36(4), pp. 246-255). Clinical manifestations include a high frequency of Raynaud's syndrome, swollen hands, sclerodactyly, arthritis, polymyositis and interstitial lung disease (Venables, (2006) Mixed connective tissue disease. Lupus, 15(3), pp. 132-137). The female to male ratio is around 3:1, and the mean age at diagnosis of adult-onset MCTD is between 35 and 40 years. MCTD is rare with point prevalence below 4 per 100,000 adults (Gunarsson et al, (2011) The prevalence and incidence of mixed connective tissue disease: a national multicentre survey of Norwegian patients. Annals of the rheumatic diseases, 70(6), pp. 1047-1051). There is currently no cure for either SjS or MCTD.

Toll like receptors (TLRs) are part of the innate immune system and recognize conserved microbial structures. TLR7 and TLR8 are expressed intracellularly in endosomes and both recognize guanosine-uridine (GU) rich single-strand RNA (ssRNA) (Heil et al, (2004) Species-specific recognition of single-stranded RNA via toll-like receptor 7 and 8. Science. 303(5663), p. 1526-1529). In several autoimmune diseases, including both SjS and MCTD, TLR7/8 are activated by self-RNA after shuttling of ribonucleoprotein (RNP) autoantigens such as Ro60 to endosomes, in the form of immune complexes with autoantibodies (Bave et al, (2005) Activation of the type I interferon system in primary Sjögren's syndrome: a possible etiopathogenic mechanism. Arthritis and rheumatism, 52(4), p. 1185-1195; Junt et al, (2015) Translating nucleic acid-sensing pathways into therapies. Nat Rev Immunol; 15:529-544). The presence of autoantibodies against another ribonucleoprotein (RNP), U1-RNP, is a diagnostic criterion for MCTD (Tani et al, (2014) The diagnosis and classification of mixed connective tissue disease. J Autoimmun. 48-49:46-9) and immune complexes of autoantibodies with U1-RNP lead to activation of TLR7 (Savarese et al (2006) U1 small nuclear ribonucleoprotein immune complexes induce type I interferon in plasmacytoid dendritic cells through TLR7. Blood, 107 (8), p. 3229-3234), and likely also TLR8.

(S)-N-(4-((5-(1,6-dimethyl-1H-pyrazolo[3,4-b]pyridin-4-yl)-3-methyl-4,5,6,7-tetrahydro-1H-pyrazolo[4,3-c]pyridin-1-yl)methyl)bicyclo[2.2.2]octan-1-yl)morpholine-3-carboxamide (WO 2018/047081) is an orally bioavailable low-molecular weight (LMW) antagonist of human TLR7 and TLR8, and is intended for the treatment of systemic autoimmune diseases, such as SjS or MCTD, where pathology is thought to be driven by excessive activation of TLR7/8 through RNP autoantigen-containing immune complexes.

There are currently no adequate therapies for SjS or MCTD. As a result, there remains a high medical need for new treatment options for SjS and MCTD patients.

### SUMMARY OF THE DISCLOSURE

The invention provides a method of treating Sjögren's Syndrome or mixed connective tissue disease in a subject in need thereof, comprising administering to the subject, a therapeutically effective amount of a compound of Formula (I):

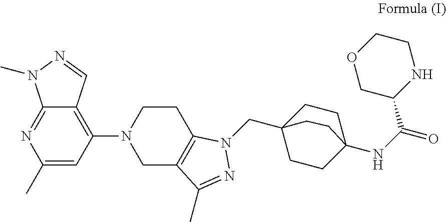 [SMILES: Cc1cc(N2CCc3c(c(C)nn3CC34CCC(NC(=O)[C@@H]5COCCN5)(CC3)CC4)C2)c2cnn(C)c2n1]

The compound of Formula (I) is also known as (S)-N-(4-((5-(1,6-dimethyl-1H-pyrazolo[3,4-b]pyridin-4-yl)-3-methyl-4,5,6,7-tetrahydro-1H-pyrazolo[4,3-c]pyridin-1-yl)methyl)bicyclo[2.2.2]octan-1-yl)morpholine-3-carboxamide. The dosing amount herein refers to the amount of the anhydrous free base of the compound of Formula (I).

### Sjören's Syndrome

Embodiment 1A: A method of treating Sjögren's Syndrome in a subject in need thereof, comprising administering, e.g. orally, to the subject a compound of Formula (I), or a pharmaceutically acceptable salt thereof, at a dose of about 25 mg to about 400 mg, e.g. about 50 mg to about 300 mg, e.g. about 100 mg to about 200 mg, twice daily, e.g. about every 12 hours.

Embodiment 2A: A method of treating Sjögren's Syndrome in a subject in need thereof, comprising administering, e.g. orally, to the subject a compound of Formula (I), or a pharmaceutically acceptable salt thereof, at a dose of about 25 mg, about 50 mg, about 75 mg, about 100 mg, about 150 mg, about 200 mg, about 250 mg, about 300 mg, or about 400 mg, twice daily, e.g. about every 12 hours.

Embodiment 3A: A method of treating Sjögren's Syndrome in a subject in need thereof, comprising administering, e.g. orally, to the subject a compound of Formula (I), or a pharmaceutically acceptable salt thereof, at a dose of about 100 mg to about 200 mg, twice daily, e.g. about every 12 hours.

Embodiment 4A: A method of treating Sjögren's Syndrome in a subject in need thereof, comprising administering, e.g. orally, to the subject a compound of Formula (I), or a pharmaceutically acceptable salt thereof, at a dose of about 250 mg, twice daily, e.g. about every 12 hours.

Embodiment 5A: A method of treating Sjögren's Syndrome in a subject in need thereof, comprising administering, e.g. orally, to the subject a compound of Formula (I), or a pharmaceutically acceptable salt thereof, at a dose of about 200 mg, twice daily, e.g. about every 12 hours.

Embodiment 6A: A method of treating Sjögren's Syndrome in a subject in need thereof, comprising administering, e.g. orally, to the subject a compound of Formula (I), or a pharmaceutically acceptable salt thereof, at a dose of about 100 mg, twice daily, e.g. about every 12 hours.

Embodiment 7A: A method of treating Sjögren's Syndrome in a subject in need thereof, comprising administering, e.g. orally, to the subject a compound of Formula (I), or a pharmaceutically acceptable salt thereof, at a dose of about 75 mg, twice daily, e.g. about every 12 hours.

Embodiment 8A: A method of treating Sjögren's Syndrome in a subject in need thereof, comprising administering, e.g. orally, to the subject a compound of Formula (I), or a pharmaceutically acceptable salt thereof, at a dose of about 50 mg, twice daily, e.g. about every 12 hours.

Embodiment 9A: A method of treating Sjögren's Syndrome in a subject in need thereof, comprising administering, e.g. orally, to the subject a compound of Formula (I), or a pharmaceutically acceptable salt thereof, at a dose of about 25 mg, twice daily, e.g. about every 12 hours.

Embodiment 10A: A method of any of Embodiments 1A to 9A, wherein the subject e.g., a patient, is positive, e.g. seropositive, for anti-extractable nuclear (ENA) antibodies, e.g. anti-Sjögren's syndrome related antigen A (anti-Ro/SSA) antibodies.

Embodiment 11A: A method of any of Embodiments 1A to 10A, wherein the subject has mild, or moderate to severe Sjögren's Syndrome.

Embodiment 12A: Use of a compound of Formula (I), or a pharmaceutically acceptable salt thereof, in the manufacture of a medicament for treating Sjögren's Syndrome in a subject, e.g. patient, in need thereof, wherein the compound of Formula (I), or a pharmaceutically acceptable salt thereof is administered according to any of Embodiments 1A to 11A.

Embodiment 13A: A compound of Formula (I), or a pharmaceutically acceptable salt thereof, for use in treating Sjögren's Syndrome, wherein the compound of Formula (I), or a pharmaceutically acceptable salt thereof is administered according to any of Embodiments 1A to 11A.

### Mixed Connective Tissue Disease

Embodiment 1B: A method of treating mixed connective tissue disease in a subject in need thereof, comprising administering, e.g. orally, to the subject a compound of Formula (I), or a pharmaceutically acceptable salt thereof, at a dose of about 25 mg to about 400 mg, e.g. about 50 mg to about 300 mg, e.g. about 100 mg to about 200 mg, twice daily, e.g. about every 12 hours.

Embodiment 2B: A method of treating mixed connective tissue disease in a subject in need thereof, comprising administering, e.g. orally, to the subject a compound of Formula (I), or a pharmaceutically acceptable salt thereof, at a dose of about 25 mg, about 50 mg, about 75 mg, about 100 mg, about 150 mg, about 200 mg, about 250 mg, about 300 mg, or about 400 mg, twice daily, e.g. about every 12 hours.

Embodiment 3B: A method of treating mixed connective tissue disease in a subject in need thereof, comprising administering, e.g. orally, to the subject a compound of Formula (I), or a pharmaceutically acceptable salt thereof, at a dose of about 100 mg to about 200 mg, twice daily, e.g. about every 12 hours.

Embodiment 4B: A method of treating mixed connective tissue disease in a subject in need thereof, comprising administering, e.g. orally, to the subject a compound of Formula (I), or a pharmaceutically acceptable salt thereof, at a dose of about 250 mg, twice daily, e.g. about every 12 hours.

Embodiment 5B: A method of treating mixed connective tissue disease in a subject in need thereof, comprising administering, e.g. orally, to the subject a compound of Formula (I), or a pharmaceutically acceptable salt thereof, at a dose of about 200 mg, twice daily, e.g. about every 12 hours.

Embodiment 6B: A method of treating mixed connective tissue disease in a subject in need thereof, comprising administering, e.g. orally, to the subject a compound of Formula (I), or a pharmaceutically acceptable salt thereof, at a dose of about 100 mg, twice daily, e.g. about every 12 hours.

Embodiment 7B: A method of treating mixed connective tissue disease in a subject in need thereof, comprising administering, e.g. orally, to the subject a compound of Formula (I), or a pharmaceutically acceptable salt thereof, at a dose of about 75 mg, twice daily, e.g. about every 12 hours.

Embodiment 8B: A method of treating mixed connective tissue disease in a subject in need thereof, comprising administering, e.g. orally, to the subject a compound of Formula (I), or a pharmaceutically acceptable salt thereof, at a dose of about 50 mg, twice daily, e.g. about every 12 hours.

Embodiment 9B: A method of treating mixed connective tissue disease in a subject in need thereof, comprising administering, e.g. orally, to the subject a compound of Formula (I), or a pharmaceutically acceptable salt thereof, at a dose of about 25 mg, twice daily, e.g. about every 12 hours.

Embodiment 10B: A method of any of Embodiments 1B to 8B, wherein the subject, e.g., a patient, is positive, e.g. seropositive, for ribonucleoproteins (RNP) anti-nuclear antibodies, e.g. anti-U1-RNP antibodies.

Embodiment 11B: Use of a compound of Formula (I), or a pharmaceutically acceptable salt thereof, in the manufacture of a medicament for treating mixed connective tissue disease in a subject, e.g. patient, in need thereof, wherein the compound of Formula (I), or a pharmaceutically acceptable salt thereof is administered according to any of Embodiments 1B to 10B.

Embodiment 12B: A compound of Formula (I), or a pharmaceutically acceptable salt thereof, for use in treating mixed connective tissue disease, wherein the compound of Formula (I), or a pharmaceutically acceptable salt thereof is administered according to any of Embodiments 1B to 10B.

### BRIEF DESCRIPTION OF THE FIGURES

FIG. 1: Time course of the compound of Formula (I) concentration in plasma after single ascending doses from 3 mg to 1000 mg.

FIG. 2: Time course of the compound of Formula (I) concentration in plasma after multiple ascending doses from 25 mg b.i.d. to 400 mg b.i.d.

FIG. 3: Food effect as observed after a dose of 200 mg of the compound of Formula (I), either in presence or in absence of standardized food.

### DETAILED DESCRIPTION OF THE DISCLOSURE

Toll-like receptor (TLR) 7 and TLR8 are endosomal receptors for GU-rich single-stranded RNA (ssRNA). In humans, TLR7 is highly expressed in interferon alpha (IFNα)-producing plasmacytoid dendritic cells (pDCs) and B cells, while TLR8 is highly expressed by monocytes, macrophages and neutrophils. TLR7/8 activation leads to monocyte and B cell activation via NFκB, as well as pDC activation via IRF7.

(S)-N-(4-((5-(1,6-dimethyl-1H-pyrazolo[3,4-b]pyridin-4-yl)-3-methyl-4,5,6,7-tetrahydro-1H-pyrazolo[4,3-c]pyridin-1-yl)methyl)bicyclo[2.2.2]octan-1-yl)morpholine-3-carboxamide, or a pharmaceutically acceptable salt thereof is referred to herein as Compound of Formula (I):

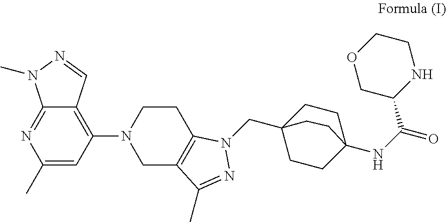 [SMILES: Cc1cc(N2CCc3c(c(C)nn3CC34CCC(NC(=O)[C@@H]5COCCN5)(CC3)CC4)C2)c2cnn(C)c2n1]

The compound of Formula (I) is highly selective for TLR7 and TLR8 over other TLRs and the IL-1 receptor on human PBMCs. The compound was described in WO 2018/047081. This compound is a selective and potent antagonist of TLR7 and TLR8, and may be used in a TLR7 or TLR8 mediated disease or disorder.

Accordingly, described herein is a dosing regimen for treating SjS patients with the compound (S)-N-(4-((5-(1,6-dimethyl-1H-pyrazolo[3,4-b]pyridin-4-yl)-3-methyl-4,5,6,7-tetrahydro-1H-pyrazolo[4,3-c]pyridin-1-yl)methyl)bicyclo[2.2.2]octan-1-yl)morpholine-3-carboxamide, or a pharmaceutically acceptable salt thereof.

Accordingly, described herein is a dosing regimen for treating MCTD patients with the compound (S)-N-(4-((5-(1,6-dimethyl-1H-pyrazolo[3,4-b]pyridin-4-yl)-3-methyl-4,5,6,7-tetrahydro-1H-pyrazolo[4,3-c]pyridin-1-yl)methyl)bicyclo[2.2.2]octan-1-yl)morpholine-3-carboxamide, or a pharmaceutically acceptable salt thereof.

Accordingly, described herein is a method of selecting the target patient population, a method of monitoring treatment of the target patient population, and a method of assessing safety and efficacy of treatment of the target patient population.

### Definitions

For purposes of interpreting this specification, the following definitions will apply and whenever appropriate, terms used in the singular will also include the plural and vice versa.

The phrase “pharmaceutically acceptable” as employed herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compound of the disclosure include, for example, isotopes of hydrogen, carbon, nitrogen, oxygen, fluorine, and chlorine, such as ³H, ¹¹C, ¹³C, ¹⁴C ¹⁵N, ¹⁸F, and ³⁶Cl. Accordingly, it should be understood that the present disclosure includes compound that incorporates one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F-labeled compound may be particularly desirable for PET or SPECT studies. Isotopically-labeled compounds can generally be prepared by conventional techniques known to those skilled in the art, e.g., using an appropriate isotopically-labeled reagent in place of the non-labeled reagent previously employed.

The term “pharmaceutical combination” as used herein means a product that results from the use or mixing or combining of more than one active ingredient. It should be understood that pharmaceutical combination as used herein includes both fixed and non-fixed combinations of the active ingredients. The term “fixed combination” means that the active ingredients, e.g., a compound of formula (I) or a pharmaceutically acceptable salt thereof, and one or more combination partners, are administered to a patient simultaneously as a single entity or dosage form. The term in such case refers to a fixed dose combination in one unit dosage form (e.g., capsule, tablet, or sachet). The terms “non-fixed combination” or a “kit of parts” both mean that the active ingredients, e.g., a compound of the present disclosure and one or more combination partners and/or one or more co-agents, are administered or co-administered to a patient independently as separate entities either simultaneously, concurrently or sequentially with no specific time limits wherein such administration provides therapeutically effective levels of the two compounds in the body of the patient, especially where these time intervals allow that the combination partners show a cooperative, e.g., an additive or synergistic effect. The term “non-fixed combination” also applies to cocktail therapy, e.g., the administration of three or more active ingredients. The term “non-fixed combination” thus defines especially administration, use, composition or formulation in the sense that the compound described herein can be dosed independently of each other, i.e., simultaneously or at different time points. It should be understood that the term “non-fixed combination” also encompasses the use of a single agent together with one or more fixed combination products with each independent formulation having distinct amounts of the active ingredients contained therein. It should be further understood that the combination products described herein as well as the term “non-fixed combinations” encompasses active ingredients (including the compounds described herein) where the combination partners are administered as entirely separate pharmaceutical dosage forms or as pharmaceutical formulations that are also sold independently of each other. Instructions for the use of the non-fixed combination are or may be provided in the packaging, e.g., leaflet or the like, or in other information that is provided to physicians and/or medical staff. The independent formulations or the parts of the formulation, products, or compositions, can then be administered simultaneously or chronologically staggered, that is the individual parts of the kit of parts can each be administered at different time points and/or with equal or different time intervals for any part of the kit of parts. Particularly, the time intervals for the dosing are chosen such that the effect on the treated disease with the combined use of the parts is larger/greater than the effect obtained by use of only compound of Formula (I); thus the compounds used in pharmaceutical combination described herein are jointly active. The ratio of the total amounts of a compound of formula I to a second agent to be administered as a pharmaceutical combination can be varied or adjusted in order to better accommodate the needs of a particular patient sub-population to be treated or the needs of the single patient, which can be due, for example, to age, sex, body weight, etc. of the patients.

The terms “co-administration” or “combined administration” or the like as utilized herein are meant to encompass the administration of one or more compounds described herein together with a selected combination partner to a single subject in need thereof (e.g., a patient or subject), and are intended to include treatment regimens in which the compounds are not necessarily administered by the same route of administration and/or at the same time.

The term “pharmaceutical composition” is defined herein to refer to a mixture (e.g., a solution or an emulsion) containing at least one active ingredient or therapeutic agent to be administered to a warm-blooded animal, e.g., a mammal or human, in order to prevent or treat a particular disease or condition affecting the warm-blooded animal.

The term “a therapeutically effective amount” of a compound (i.e. compound of Formula (I) or a pharmaceutically acceptable salt thereof) of the present disclosure refers to an amount of the compound of the present disclosure that will elicit the biological or medical response of a subject (patient or subject), for example, reduction or inhibition of an enzyme or a protein activity, or ameliorate symptoms, alleviate conditions, slow or delay disease progression, or prevent a disease, etc. The therapeutically effective dosage of a compound, the pharmaceutical composition, or the combinations thereof, is dependent on the ethnicity of the patient, the body weight, age, sex, and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

As used herein, the term “carrier” or “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

As used herein, the term “subject” refers to an animal. Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In a preferred embodiment, the subject is a human. The term “subject” is used interchangeably with “patient” when it refers to human.

As used herein, a subject is “in need of” a treatment if such subject would benefit biologically, medically or in quality of life from such treatment.

As used herein, the phrase “population of patients” is used to mean a group of patients.

The term “comprising” encompasses “including” as well as “consisting,” e.g., a composition “comprising” X may consist exclusively of X or may include something additional, e.g., X+Y.

The term “about” in relation to a numerical value x means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The term “treatment” or “treat” is herein defined as the application or administration of a compound according to the disclosure, (compound of Formula (I), or a pharmaceutically acceptable salt thereof, or a pharmaceutical composition comprising said compound, to a subject or to an isolated tissue or cell line from a subject, where the subject has a particular disease (e.g., SjS), a symptom associated with the disease (e.g., SjS), or a predisposition towards development of the disease (e.g., SjS) (if applicable), where the purpose is to cure (if applicable), delay the onset of, reduce the severity of, alleviate, ameliorate one or more symptoms of the disease, improve the disease, reduce or improve any associated symptoms of the disease or the predisposition toward the development of the disease. The term “treatment” or “treat” includes treating a patient suspected to have the disease as well as patients who are ill or who have been diagnosed as suffering from the disease or medical condition, and includes suppression of clinical relapse.

As used herein, “selecting” and “selected” in reference to a patient is used to mean that a particular patient is specifically chosen from a larger group of patients on the basis of (due to) the particular patient having a predetermined criteria. Similarly, “selectively treating” refers to providing treatment to a patient having a particular disease, where that patient is specifically chosen from a larger group of patients on the basis of the particular patient having a predetermined criterion. Similarly, “selectively administering” refers to administering a drug to a patient that is specifically chosen from a larger group of patients on the basis of (due to) the particular patient having a predetermined criterion. By “selecting”, “selectively treating” and “selectively administering”, it is meant that a patient is delivered a personalized therapy based on the patient's personal history (e.g., prior therapeutic interventions, e.g., prior treatment with biologics), biology (e.g., particular genetic markers), and/or manifestation (e.g., not fulfilling particular diagnostic criteria), rather than being delivered a standard treatment regimen based solely on the patient's membership in a larger group. Selecting, in reference to a method of treatment as used herein, does not refer to fortuitous treatment of a patient having a particular criterion, but rather refers to the deliberate choice to administer treatment to a patient based on the patient having a particular criterion. Thus, selective treatment/administration differs from standard treatment/administration, which delivers a particular drug to all patients having a particular disease, regardless of their personal history, manifestations of disease, and/or biology. In some embodiments, the patient was selected for treatment based on having SjS. In some embodiments, the patient was selected for treatment based on having MCTD.

### Method of Use in Sjören's Syndrome

The present invention provides a method of treating SjS in a subject, e.g., a patient, in need thereof, comprising the step of administering, e.g. orally, to the subject, e.g., a patient, a compound of Formula (I), or a pharmaceutically acceptable salt thereof, e.g., the compound of Formula (I) hemiheptahydrate, at a dose of about 25 mg to about 400 mg, e.g. about 50 mg to about 300 mg, e.g. about 100 mg to about 200 mg, twice daily, e.g. about every 12 hours. The dosing amount refers to the amount of the anhydrous free from of the compound of Formula (I).

The present invention provides a compound of Formula (I), or a pharmaceutically acceptable salt thereof, for use in treating SjS, wherein the compound of Formula (I), or a pharmaceutically acceptable salt thereof, e.g., the compound of Formula (I) hemiheptahydrate, is administered at a dose of about 25 mg to about 400 mg, e.g. about 50 mg to about 300 mg, e.g. about 100 mg to about 200 mg, twice daily, e.g. about every 12 hours. The dosing amount refers to the amount of the anhydrous free from of the compound of Formula (I).

The present invention provides use of a compound of Formula (I), or a pharmaceutically acceptable salt thereof, in the treatment of SjS in a subject, e.g. patient, in need thereof, wherein the compound of Formula (I), or a pharmaceutically acceptable salt thereof, e.g., the compound of Formula (I) hemiheptahydrate, is administered at a dose of about 25 mg to about 400 mg, e.g. about 50 mg to about 300 mg, e.g. about 100 mg to about 200 mg, twice daily, e.g. about every 12 hours. The dosing amount refers to the amount of the anhydrous free from of the compound of Formula (I).

The present invention provides use of a compound of Formula (I), or a pharmaceutically acceptable salt thereof, in the manufacture of a medicament for the treatment of SjS in a subject, e.g. patient, in need thereof, wherein the compound of Formula (I), or a pharmaceutically acceptable salt thereof, e.g., the compound of Formula (I) hemiheptahydrate, is administered at a dose of about 25 mg to about 400 mg, e.g. about 50 mg to about 300 mg, e.g. about 100 mg to about 200 mg, twice daily, e.g. about every 12 hours. The dosing amount refers to the amount of the anhydrous free from of the compound of Formula (I).

In one embodiment of any of the inventions described above, the dose is about 25 mg, about 50 mg, about 60 mg, about 75 mg, about 90 mg, about 100 mg, about 150 mg, about 200 mg, about 250 mg, about 300 mg, about 400 mg, twice daily, e.g. about every 12 hours.

In one embodiment of any of the inventions described above, the dose is about 200 mg, twice daily, e.g. about every 12 hours.

In one embodiment of any of the inventions described above, the subject, e.g., a patient, is positive, e.g. seropositive, for anti-extractable nuclear (ENA) antibodies, e.g. anti-Sjögren's syndrome related antigen A (anti-Ro/SSA) antibodies.

In one embodiment of any of the inventions described above, the subject, e.g., a patient, has mild SjS.

In one embodiment of any of the inventions described above, the subject, e.g., a patient, has moderate to severe SjS.

### Method of Use in Mixed Connective Tissue Disease

The present invention provides a method of treating MCTD in a subject, e.g., a patient, in need thereof, comprising the step of administering, e.g. orally, to the subject, e.g., a patient, a compound of Formula (I), or a pharmaceutically acceptable salt thereof, e.g., the compound of Formula (I) hemiheptahydrate, at a dose of about 25 mg to about 400 mg, e.g. about 50 mg to about 300 mg, e.g. about 100 mg to about 200 mg, twice daily, e.g. about every 12 hours. The dosing amount refers to the amount of the anhydrous free from of the compound of Formula (I).

The present invention provides a compound of Formula (I), or a pharmaceutically acceptable salt thereof, for use in treating MCTD, wherein the compound of Formula (I), or a pharmaceutically acceptable salt thereof, e.g., the compound of Formula (I) hemiheptahydrate, is administered at a dose of about 25 mg to about 400 mg, e.g. about 50 mg to about 300 mg, e.g. about 100 mg to about 200 mg, twice daily, e.g. about every 12 hours. The dosing amount refers to the amount of the anhydrous free from of the compound of Formula (I).

The present invention provides use of a compound of Formula (I), or a pharmaceutically acceptable salt thereof, in the treatment of MCTD in a subject, e.g. patient, in need thereof, wherein the compound of Formula (I), or a pharmaceutically acceptable salt thereof, e.g., the compound of Formula (I) hemiheptahydrate, is administered at a dose of about 25 mg to about 400 mg, e.g. about 50 mg to about 300 mg, e.g. about 100 mg to about 200 mg, twice daily, e.g. about every 12 hours. The dosing amount refers to the amount of the anhydrous free from of the compound of Formula (I).

The present invention provides use of a compound of Formula (I), or a pharmaceutically acceptable salt thereof, in the manufacture of a medicament for the treatment of mixed connective tissue disease in a subject, e.g. patient, in need thereof, wherein the compound of Formula (I), or a pharmaceutically acceptable salt thereof, e.g., the compound of Formula (I) hemiheptahydrate, is administered at a dose of about 25 mg to about 400 mg, e.g. about 50 mg to about 300 mg, e.g. about 100 mg to about 200 mg, twice daily, e.g. about every 12 hours. The dosing amount refers to the amount of the anhydrous free from of the compound of Formula (I).

In one embodiment of any of the inventions described above, the dose is about 25 mg, about 50 mg, about 60 mg, about 75 mg, about 90 mg, about 100 mg, about 150 mg, about 200 mg, about 250 mg, about 300 mg, about 400 mg, twice daily, e.g. about every 12 hours.

In one embodiment of any of the inventions described above, the dose is about 200 mg, twice daily, e.g. about every 12 hours.

In one embodiment of any of the inventions described above, the subject, e.g., a patient, is positive, e.g. seropositive, for anti-extractable nuclear (ENA) antibodies, e.g. anti-U1-RNP antibodies.

### Effectiveness of Treatment of Sjören's Syndrome and MCTD

The disclosed TLR7 and TLR8 antagonist, i.e., compound of Formula (I), or a pharmaceutically acceptable salt thereof, may be used in vitro, ex vivo, or incorporated into pharmaceutical compositions and administered in vivo to treat SjS patients (e.g., human patients).

The disclosed TLR7 and TLR8 antagonist, i.e., compound of Formula (I), or a pharmaceutically acceptable salt thereof, may be used in vitro, ex vivo, or incorporated into pharmaceutical compositions and administered in vivo to treat MCTD patients (e.g., human patients).

The effectiveness of a SjS treatment or MCTD treatment may be assessed using various known methods and tools that measure the state and/or clinical response for the respective disease. Some examples include, e.g., EULAR Sjögren's Syndrome Disease Activity Index (ESSDAI), Physician Global Assessment Scale (PhGA), EULAR Sjögren's Syndrome Patient Reported Index (ESSPRI), and The Functional Assessment of Chronic Illness Therapy-Fatigue Scale (FACIT-Fatigue).

### Efficacy

Clinical efficacy measurements related to primary and secondary objectives are outlined below.

### EULAR Sjögren's Syndrome Disease Activity Index (ESSDAI)

ESSDAI is a validated disease outcome measure for SjS and is applied to the study subjects (Seror R, et al (2015) Validation of EULAR primary Sjögren's syndrome disease activity (ESSDAI) and patient indexes (ESSPRI). Ann. Rheum. Dis. p. 859-66). The instrument contains 12 organ-specific domains contributing to disease activity. For each domain, features of disease activity are scored in 3 or 4 levels according to their severity. These scores are then summed across the 12 domains in a weighted manner to provide the total score. The domains (weights) are as follows: constitutional (3), lymphadenopathy (4), glandular (2), articular (2), cutaneous (3), pulmonary (5), renal (5), muscular (6), PNS (5), CNS (5), hematological (2), and biological (1). The maximum possible score is 123.

In our study, to calculate ESSDAI, all 12 organ domains must be individually assessed at every scheduled timepoint (from screening visit till end of study). Domain assessments are entered into a table (provided by a central vendor) and ESSDAI score is calculated by the software.

For assessments not listed in the protocol as mandatory tests but which may be needed to estimate ESSDAI, including radiography, high resolution computer tomography (HRCT), lung function test (DLCO, FVC), estimated glomerular filtration rate (eGFR), electromyography (EMG), muscle (or any other) biopsy, it is at the investigator's discretion to have these assessed based on the signs and symptoms of the patient so to provide correct ESSDAI readout.

Participants with MCTD will complete the articular (from 0 “no activity” to 3 “high activity”) and pulmonary (from 0 “no activity to 3 “high activity”) domains of the ESSDAI only. These domains will be assessed separately over the treatment period.

### Physician Global Assessment Scale (PhGA) (SjS and MCTD Participants)

The physician's global assessment scale is used by the Investigator to rate the disease activity of their patient using 100 mm VAS ranging from “no disease activity” (0) to “maximal disease activity” (100).

To enhance objectivity, the physician must not be aware of the specific patient's reported outcome assessments, when performing his own assessment on that patient. Therefore this assessment must be done prior to viewing the patient's reported outcomes.

### FACIT-Fatigue (SjS and MCTD Participants)

The Functional Assessment of Chronic Illness Therapy-Fatigue Scale (FACIT-F v4) is a short, 13-item, easy-to-administer tool that measures an individual's level of fatigue during their usual daily activities over the past week. The level of fatigue is measured on a 5-point Likert scale (0=not at all, 1=a little bit, 2=somewhat, 3=quite a bit, 4=very much) (Webster et al 2003).

### EULAR Sjögren's Syndrome Patient Reported Index (ESSPRI) (SjS Participants)

ESSPRI is an established disease outcome measure for Sjögren's Syndrome (Seror R, et al (2011) EULAR Sjögren's Syndrome Patient Reported Index (ESSPRI): development of a consensus patient index for primary Sjögren's syndrome. Ann. Rheum. Dis. p. 968-72). It consists of three of domains of dryness, pain and fatigue. The subject can assess severity of symptoms they experience on a single 0-10 numerical scale for each of the three domains. The ESSPRI score is defined as mean of scores from the three scales: (dryness+pain+fatigue)/3.

### Schirmer's Test (SjS Participants)

Schirmer's test is used to measure the quantity of tear secretion especially for those who suffer from dry eye syndrome.

The participant is seated in the examining chair with the room lights dimmed and their head against a headrest for comfort. Both eyes will be assessed simultaneously.

The procedure is briefly outlined below:
The eye is gently dried of excess tears
The Schirmer strip is folded 5 mm from one end and kept in the lower fornix at the junction of lateral ⅓ and medial ⅔ (do not touch cornea or lashes)
The participant is asked to close the eyes
Tears in the conjunctival sac will cause progressive wetting of the paper strip.
After 5 minutes, the filter paper is removed and the distance between the leading edge of wetness and the initial fold is measured, using a millimeter ruler

### Salivary Flow Rate (Unstimulated) (SjS Participants)

Unstimulated whole salivary fluid is obtained from participants at Screening, Baseline, Week 12 and Week 24.

Unstimulated salivary secretions are collected over 5 minutes. All assessments are to be performed at a fixed time of the day to minimize fluctuations related to the circadian rhythm of salivary flow and composition. Participants are instructed not to eat, drink or smoke for 90 minutes before the assessment. The start time and end time of saliva collection will be recorded to calculate the salivary flow rate per minute.

### Forced Vital Capacity (FVC) and Forced Expiratory Volume (FEV) (MCTD Participants)

Forced expiratory volume (FEV) measures how much air a person can exhale during a forced breath. The amount of air exhaled may be measured during the first (FEV1), second (FEV2), and/or third seconds (FEV3) of the forced breath. Forced vital capacity (FVC) is the total amount of air exhaled during the FEV test. Forced expiratory volume and forced vital capacity are lung function tests that are measured during spirometry, and are important measures of lung function.

### Diffusing Capacity of the Lungs for Carbon Monoxide (DLCO) (MCTD Participants)

DLCO is a measurement to assess the ability of the lungs to transfer gas from inspired air to the bloodstream. Inhaled carbon monoxide (CO) is used for this test due to its high affinity for hemoglobin. In brief, during a ten-second breath-hold, DLCO measures uptake of CO per time per CO pressure (cc of CO/see/mm of Hg; (Ogilvie et al 1957; Cotes et al 1993; Modi et al 2020).

### Raynaud's Condition Score (RCS) (MCTD Participants)

The Raynaud's Condition score (RCS) is participant's rating of difficulty considering number of attacks, duration, amount of pain, numbness, or other symptoms caused in the fingers (including painful sores) due to the Raynaud's phenomenon every day and impact of Raynaud's alone on use of hands every day. An 11 point Likert scale is used to rate the difficulty caused by the condition each day with 0=no difficulty and 10=extreme difficulty. Participants are asked to select the number that best describes their difficulty, with higher score indicating worse condition.

### King's Brief Interstitial Lung Disease (K-BILD) (MCTD Participants)

The K-BILD questionnaire is a self-administered health-status questionnaire that has been developed in patients with interstitial lung diseases. It consists of 15 items in three domains: i) breathlessness and activities, ii) psychological factors, and iii) chest symptoms. Domain and total scores range from 0 to 100, with higher scores representing better health status (Patel et al 2013; Sinha et al 2019; Nolan et al 2019; Flaherty et al 2019).

### Efficacy Assessments

Efficacy measures in this study are primarily based on ESSDAI (EULAR SS Disease Activity Index) measuring organ-specific disease criteria, and on ESSPRI (European League Against Rheumatism [EULAR] Sjögren's Syndrome [SS] Patient Reported Index) measuring the patient's subjective disease impact. Both instruments are widely accepted and validated, gold-standard measures of systemic and symptomatic manifestations of SjS, respectively.

ESSDAI is a systemic disease activity index that classifies disease activity in 3-4 levels, over each of 12 differentially weighted domains (biologic, hematologic, articular, glandular, cutaneous, constitutional, lymphadenopathy, renal, pulmonary, PNS, CNS and muscular). A composite weighted score provides an accurate assessment of disease activity, with a good sensitivity to change, as validated in multiple cohort studies (Seror R et al (2015) Validation of EULAR primary Sjögren's syndrome disease activity (ESSDAI) and patient indexes (ESSPRI). Ann. Rheum. Dis. p. 859-66). The ESSPRI tool, on the other hand, is a patient reported composite score of symptoms of dryness, limb pain and fatigue evaluated on 0-10 visual analog scale, during the preceeding 2 weeks (Seror R et al (2011) EULAR Sjögren's Syndrome Patient Reported Index (ESSPRI): development of a consensus patient index for primary Sjögren's syndrome. Ann. Rheum. Dis. p. 968-72). Patient reported scores have poor sensitivity to change in disease activity, but among available tools, ESSPRI has been reported to have significantly better sensitivity. A recent prospective study reported poor correlation between systemic and patient scores, suggesting that the two indices evaluate complementary components of disease activity, therefore underscoring the importance of evaluation of both parameters to arrive at an accurate assessment of disease activity and change thereof (Seror R et al (2015) Validation of EULAR primary Sjögren's syndrome disease activity (ESSDAI) and patient indexes (ESSPRI). Ann. Rheum. Dis. p. 859-66).

### Pharmaceutical Composition

The TLR7/8 antagonist, i.e., compound of Formula (I), or a pharmaceutically acceptable salt thereof, may be used as a pharmaceutical composition when combined with a pharmaceutically acceptable carrier. Such a composition may contain, in addition to the compound of Formula (I), carriers, various diluents, fillers, salts, buffers, stabilizers, solubilizers, and other materials known in the art. The characteristics of the carrier depends on the route of administration. The pharmaceutical compositions for use in the disclosed methods may also contain additional therapeutic agents for treatment of the particular targeted disorder. For example, a pharmaceutical composition may also include anti-inflammatory or anti-itch agents. Such additional factors and/or agents may be included in the pharmaceutical composition to produce a synergistic effect with the compound of Formula (I), or to minimize side effects caused by the compound of Formula (I). In preferred embodiments, the pharmaceutical composition for use in the disclosed methods comprise compound of Formula (I) in a dose of about 10 mg, about 20 mg, about 25 mg, about 50 mg, about 100 mg, or about 200 mg.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients, which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Pharmaceutical compositions for use in the disclosed methods may be manufactured in conventional manner. In one embodiment, the pharmaceutical composition is provided for oral administration. For example the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

### Combinations

In practicing some of the methods of treatment or uses of the present disclosure, a therapeutically effective amount of a compound of Formula (I) or a pharmaceutically acceptable salt thereof, is administered to a patient, e.g., a mammal (e.g., a human). While it is understood that the disclosed methods provide for the treatment of SjS or MCTD patients using the compound of Formula (I) or a pharmaceutically acceptable salt thereof, the therapy is not necessarily a monotherapy. Indeed, if a patient is selected for the treatment with a compound of Formula (I), then the compound of Formula (I) or a pharmaceutically acceptable salt thereof, may be administered in accordance with the methods of the disclosure either alone or in combination with other agents and therapies for treating SjS or MCTD patients, e.g., in combination with at least one additional agent for treating SjS or MCTD. When co-administered with one or more additional agent(s) for treating SjS or MCTD, a compound of Formula (I) or a pharmaceutically acceptable salt thereof, may be administered either simultaneously with the other agent, or sequentially. If administered sequentially, the attending physician will decide on the appropriate sequence of administering the compound of Formula (I) or a pharmaceutically acceptable salt thereof, in combination with other agents and the appropriate dosages for co-delivery.

Various therapies may be beneficially combined with the disclosed compound of Formula (I) or a pharmaceutically acceptable salt thereof, during treatment of SjS or MCTD. Such therapies include steroids (corticosteroid such as prednisone or equivalent); antimalarials, for example, hydroxychloroquine; disease-modifying antirheumatic drugs (DMARDSs), for example, methotrexate, sulfasalazine, minocycline and leflunomide; or B-cell depleting drug such as Rituximab.

### Kits of the Invention

The disclosure also encompasses kits for treating SjS or MCTD. Such kits comprise a TLR7/8 antagonist, e.g., (S)-N-(4-((5-(1,6-dimethyl-1H-pyrazolo[3,4-b]pyridin-4-yl)-3-methyl-4,5,6,7-tetrahydro-1H-pyrazolo[4,3-c]pyridin-1-yl)methyl)bicyclo[2.2.2]octan-1-yl)morpholine-3-carboxamide (see Formula (I)) or a pharmaceutical composition thereof. Additionally, such kits may comprise instructions for use.

In one embodiment, the kit comprises two or more separate pharmaceutical compositions, at least one of which contains a compound of Formula (I) or a pharmaceutically acceptable salt thereof. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of Formula (I) or a pharmaceutically acceptable salt thereof, and the other agents for treating SjS or MCTD (as defined herein) may be manufactured and/or formulated by the same or different manufacturers. Moreover, the compound of Formula (I) or a pharmaceutically acceptable salt thereof, and the other agents for treating SjS or MCTD may be brought together into a combination therapy: (i) prior to release of the combination product to physicians (e.g. in the case of a kit comprising a compound of Formula (I) or a pharmaceutically acceptable salt thereof, and the other agents for treating SjS or MCTD); (ii) by the physician themselves (or under the guidance of the physician) shortly before administration; (iii) in the patient themselves, e.g. during sequential administration of the compound of Formula (I) or a pharmaceutically acceptable salt thereof, and the other agents for treating SjS or MCTD.

### Additional Embodiments

In another embodiment of the disclosed methods, uses and kits, the compound of Formula (I), or a pharmaceutically acceptable salt thereof, is administered to a patient with SjS or MCTD, in a dose of about 10 mg, about 20 mg, about 25 mg, about 35 mg, about 50 mg, about 60 mg, about 75 mg, about 90 mg, about 100 mg, about 150 mg, about 200 mg, or about 250 mg, twice daily.

In another embodiment of the disclosed methods, uses and kits, the compound of Formula (I), or a pharmaceutically acceptable salt thereof, is administered in a dose of about 100 mg twice daily.

In another embodiment of the disclosed methods, uses and kits, the compound of Formula (I), or a pharmaceutically acceptable salt thereof, is administered in a dose of about 150 mg twice daily.

In another embodiment of the disclosed methods, uses and kits, the compound of Formula (I), or a pharmaceutically acceptable salt thereof, is administered in a dose of about 200 mg twice daily.

In another embodiment of the disclosed methods, uses and kits, the patient has mild, or moderate to severe SjS. A patient with moderate to severe SjS is defined as a patient having prior to treatment with the compound of Formula (I) or a pharmaceutically acceptable salt thereof, an ESSDAI score ≥5 (i.e. at least 5) from 8 defined domains (biologic, hematologic, articular, cutaneous, glandular, lymphadenopathy, renal, constitutional) and an ESSPRI score of at least 5.

In another embodiment of the disclosed methods, uses and kits, the patient is an adult.

In another embodiment of the disclosed methods, uses and kits, the patient achieves by week 12 or by week 24 of treatment of SjS a change from baseline on at least one of the patient and/or physician-reported outcomes (i.e. ESSPRI, FACIT-F, EQ-5D, PhGA).

In another embodiment of the disclosed methods, uses and kits, the patient achieves by week 12 or by week 24 of treatment of SjS a reduction of the ESSDAI score.

In another embodiment of the disclosed methods, uses and kits, the patient achieves by week 12 or by week 24 of treatment of SjS a change from baseline of the ESSDAI score.

In another embodiment of the disclosed methods, uses and kits, the patient achieves by week 12 or by week 24 of treatment of MCTD a change from baseline on at least one of the patient and/or physician-reported outcomes (i.e. ESSPRI, FACIT-F, EQ-5D, PhGA).

In another embodiment of the disclosed methods, uses and kits, the patient achieves by week 12 or by week 24 of treatment of MCTD a reduction of the PhGA score.

In another embodiment of the disclosed methods, uses and kits, the patient achieves by week 12 or by week 24 of treatment of MCTD a change from baseline of the PhGA score.

In another embodiments of the disclosed methods, uses and kits, the patient is an adult. In some embodiments of the disclosed methods, uses and kits, the patient is an adolescent.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural references unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference. The following Examples are presented in order to more fully illustrate the preferred embodiments of the disclosure. These examples should in no way be construed as limiting the scope of the disclosed subject matter, as defined by the appended claims.

### EXAMPLES

Abbreviations

| | |
|---|---|
| AE | adverse event |
| AUC | area under the curve |
| AUCinf | area under the plasma (or serum or blood) concentration-time curve from |
| | time zero to infinity (mass × time/volume) |
| AUClast | area under the plasma (or serum or blood) concentration-time curve from |
| | time zero to time of last quantifiable concentration (mass × time/volume) |
| AUCtau | area under the plasma (or serum or blood) concentration-time curve from |
| | time zero to the end of the dosing interval tau (mass × time/volume) |
| Bid or b.i.d. | twice a day (for Latin: “bis in die”) |
| BMI | Body Mass Index |
| CBC | complete blood count |
| cm | centimeter |
| CL/F | the apparent systemic (or total body) clearance from plasma (or serum or |
| | blood) following administration (mass/volume) |
| CNS | central nervous system |
| CV | coefficient of variation |
| DMARDs | disease-modifying antirheumatic drugs |
| ECG | Electrocardiogram |
| eGFR | estimated glomerular filtration rate |
| ELISA | Enzyme-linked immunosorbent assay |
| EMG | electromyography |
| EQ-5D | EuroQual 5 dimensions (Standard instrument to measure the health- |
| | related quality of life) |
| ESSDAI | EULAR Sjögren's Syndrome Disease Activity Index |
| ESSPRI | EULAR Sjögren's Syndrome Patient Reported Index |
| EULAR | European League against Rheumatism |
| FACIT-F | Functional Assessment of Chronic Illness Therapy-Fatigue |
| FIH | First in Human |
| h | hour |
| HRCT | high resolution computer tomography |
| i.v. | intravenous |
| IA | Interim analysis |
| INR | International Normalized Ratio |
| IFNα | Interferon alpha |
| kg | kilogram |
| LC-MS/MS | liquid chromatography/mass spectrometry-mass spectrometry |
| mAb | monoclonal antibody |
| MCP-Mod | Multiple Comparison Procedure - Modelling |
| MCTD | Mixed Connective Tissue Disease |
| MMRM | Mixed effect Model Repeat Measurement |
| MRT | mean residence time |
| NOAC | Novel Oral Anti-Coagulant |
| NSAID | Nonsteroidal Anti-Inflammatory Drug |
| PD | Pharmacodynamic(s) |
| PhGA | Physician global assessment scale |
| PK | Pharmacokinetic(s) |
| PNS | peripheral nervous system |
| PT | prothrombin time |
| PTT | partial thromboplastin time |
| Qd or q.d. | once a day (for Latin “quaque die”) |
| QTcF | QT interval corrected by Fridericia's formula |
| Racc | Ratio of accumulation of drug |
| RNP | Ribonucleoprotein |
| SAE | serious adverse event |
| SjS | Sjögren's Syndrome |
| SOM | Site Operations Manual |
| SPT | skin prick test |
| SS | Safety Set |
| TLR | Toll-like receptor |
| Vz/F | the apparent volume of distribution during the terminal elimination phase |
| | following administration (volume) |

### Example 1: Phase 1 Clinical Trial

A first-in-human (FIH) study was conducted to assess the safety and tolerability, pharmacokinetics (PK) and pharmacodynamics (PD) of single and multiple doses of compound of Formula (I) as twice daily (bid) oral administration in healthy volunteers, to support further clinical development of compound of Formula (I) in autoimmune diseases. This study also explored the effect of food intake.

First-in-human study in healthy volunteers (HVs) included the following:
Part A was a randomized, subject-blinded, placebo-controlled, single ascending dose (SAD) study with 10 cohorts. Each cohort received in an ascending order a 1 mg, 3 mg, 10 mg, 20 mg, 40 mg, 80 mg, 160 mg, 320 mg, 640 mg or 1000 mg single dose of compound of Formula (I).
Part B was a randomized, subject-blinded, placebo-controlled, multiple ascending dose (MAD) study with 5 cohorts. Each cohort received in an ascending order a 25 mg, 50 mg, 100 mg, 200 mg or 400 mg dose of compound of Formula (I) b.i.d. for 14 days.
Part C was a randomized, open-label, two-sequence, two-period crossover study with a 200 mg dose of compound of Formula (I) administered each underfed or fasted condition, for the food effect evaluation.

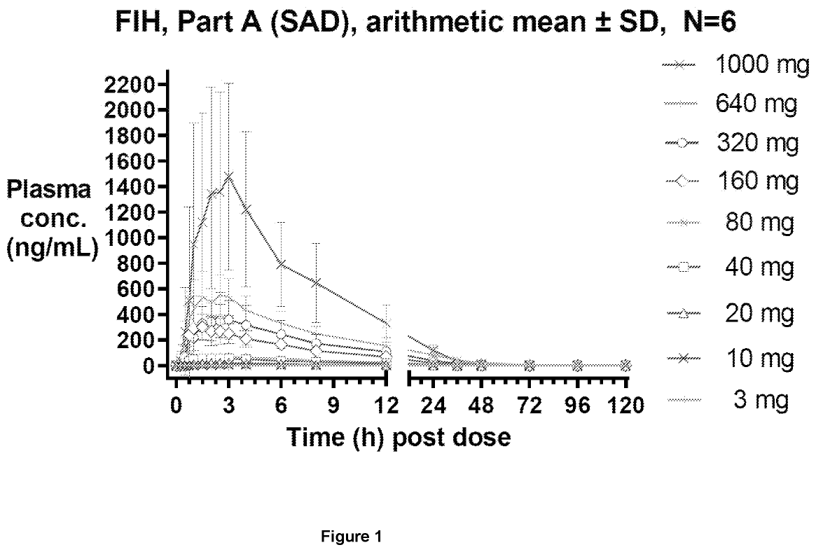

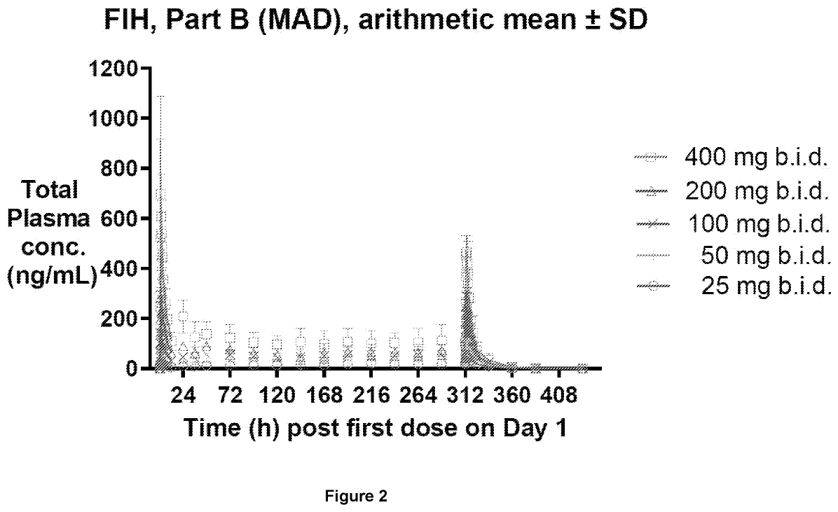

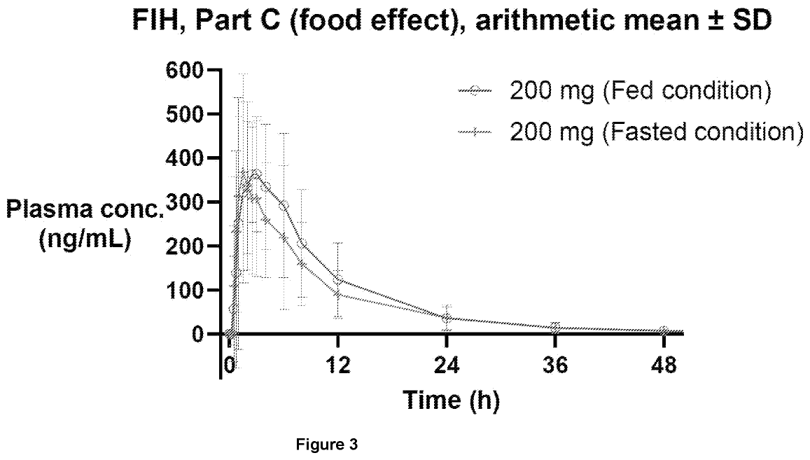

In the FIH study, an increase of exposure was observed in the single ascending dose (FIG. 1, dose-proportional for the higher doses) as well as the multiple ascending dose parts of the study (FIG. 2, dose-proportional at steady state for the lower doses). No relevant food effect was observed with the expected exception of a slightly delayed Cmax underfed condition (FIG. 3).

A single oral administration of up to 1000 mg and multiple administrations of up to 400 mg b.i.d. of the compound of Formula (I) for 14 days were safe and well tolerated by healthy subjects.

CD69 and TNF are biomarkers intended to inform the pharmacodynamic (PD) effect of compound of Formula (I). Whole blood samples from the SAD and MAD cohorts were ex-vivo stimulated with a TLR7/8 agonist to increase levels of CD69 and TNF. Across the dose range, compound of Formula (I) inhibited B cell activation following TLR7 stimulation, as compound of Formula (I) blocked the upregulation of activation markers such as CD69. In addition, Compound of Formula (I) inhibited activation of human monocytes to produce TNF following TLR8 stimulation across the dose range.

### Example 2: Efficacy and Safety in Subjects with Sjögren's Syndrome or Mixed Connective Tissue Disease

A Phase 2 study is conducted with the compound of Formula (I) designed to establish safety, tolerability and efficacy of the compound of Formula (I) in subjects with SjS or MCTD to allow further development of the compound for treatment of these diseases.

Protocol Summary

| | |
|---|---|
| Brief Title | A study to evaluate the safety, tolerability and efficacy of the |
| | compound of Formula (I) in participants with Sjögren's Syndrome |
| | (SjS) or Mixed Connective Tissue Disease (MCTD) |
| Study Type | Interventional |
| Purpose | This study with compound of Formula (I) is designed to establish |
| | safety, tolerability and efficacy of the compound of Formula (I) in |
| | SjS and MCTD. |
| Primary Objective(s) | SjS: |
| | To evaluate the efficacy of the compound of Formula (I) |
| | compared to placebo based on change from baseline in ESSDAI at |
| | Week 24. |
| | MCTD: |
| | To evaluate the efficacy of the compound of Formula (I) |
| | compared to placebo based on change from baseline in Physician |
| | Global Assessment (PhGA) at Week 24. |
| Secondary | SjS and MCTD: |
| Objectives | To evaluate the efficacy of the compound of Formula (I) |
| | compared to placebo based on change from baseline on |
| | participant and physician-reported outcomes over time up to |
| | Week 24. |
| | To evaluate the safety and tolerability of compound of |
| | Formula (I). |
| | To assess PK parameters of the compound of Formula (I) |
| | (Cmax, AUC, Tmax and others) at steady state. |
| | SjS: |
| | To explore the effect of the compound of Formula (I) on |
| | quantitative salivary flow (unstimulated) over 24 weeks. |
| | To explore the effect of the compound of Formula (I) on |
| | quantitative tear production over 24 weeks. |
| | MCTD: |
| | To evaluate the efficacy of the compound of Formula (I) |
| | based on change from baseline in Forced Vital Capacity |
| | (FVC) and Forced Expiratory Volume (FEV1, FEV2, FEV3) |
| | over time up to Week 24. |
| | To evaluate the efficacy of the compound of Formula (I) |
| | based on change from baseline in the diffusing capacity of |
| | lungs for carbon monoxide (DLCO) over time up to Week 24. |
| | To evaluate the efficacy of the compound of Formula (I) |
| | based on change from baseline in the patient reported |
| | outcome on lung function. |
| | To evaluate the efficacy of the compound of Formula (I) |
| | compared to placebo based on change from baseline in |
| | Raynaud's Condition Score (RCS) over time up to Week 24. |
| Study Design | This is a randomized, participant and investigator-blinded, placebo- |
| | controlled, multi center parallel group basket study to evaluate the |
| | safety, tolerability and efficacy of 200 mg twice daily (b.i.d) of the |
| | compound of Formula (I) in participants with SjS or with MCTD over |
| | 24 weeks of treatment, followed by 4 weeks of follow-up. Total study |
| | duration for each participant will be up to 34 weeks. |
| Population | Female and male participants aged 18 to 75 years with moderate to |
| | severe SjS or diagnosis of MCTD; a total of approximately 60 |
| | participants will be enrolled in this study: approximately 48 |
| | participants with SjS and approximately 12 participants with MCTD. |
| Key Inclusion | SjS and MCTD: |
| Criteria | Male or female participants aged 18 to 75 years at |
| | screening |
| | Fully vaccinated with a locally approved COVID-19 |
| | vaccination at least 4 weeks prior to baseline |
| | SjS: |
| | Classification of SjS according to the 2016 |
| | ACR/EULAR criteria at screening |
| | Screening ESSDAI (based on weighted score) ≥5 from 8 |
| | defined domains (biologic, hematologic, articular, |
| | cutaneous, glandular, lymphadenopathy, renal, |
| | constitutional). Participants with involvement of one or more |
| | of the remaining 4 domains are eligible but scores of these |
| | domains will not contribute to the assessment for eligibility, |
| | but will be part of the overall ESSDAI score for that subject |
| | Seropositive for anti-Ro/SSA antibodies at screening |
| | Unstimulated whole salivary flow rate of ≥0 mL/min at |
| | screening |
| | MCTD: |
| | Diagnosis of MCTD based on modified Kahn's criteria |
| | Serological criteria: seropositive for anti-U1-RNP |
| | antibodies at central lab |
| | Raynaud's phenomenon |
| | At least two of the four following signs: i) synovitis, ii) |
| | myositis, iii) swollen fingers and vi) interstitial lung |
| | disease |
| | Patients with overlap syndromes, i.e. patients meeting |
| | diagnostic criteria for systemic autoimmune disease other |
| | than MCTD (e.g. SLE, scleroderma, dermato- or |
| | polimyositis, rheumatoid arthritis or SjS) may be included |
| | unless they have major organ involvement (e.g. lupus |
| | nephritis) as judged by the investigator |
| Key Exclusion | SjS and MCTD: |
| Criteria | Rituximab or other B cell depleting drug within 6 months of |
| | baseline. For participants who received B cell depleting therapy |
| | within 6-12 months, B cell count should be within normal range. |
| | Prior treatment with any of the following within 3 months of |
| | baseline |
| | CTLA4-Fc Ig (abatacept) |
| | Anti-TNF mAb |
| | Intravenous Ig |
| | Plasmapheresis |
| | i.v. or oral cyclophosphamide |
| | i.v. or oral cyclosporine A |
| | Screening CBC laboratory values as follows: |
| | Hemoglobin levels below 8 g/dL |
| | Total leukocyte count less than 2,000/μL |
| | Platelets less than 50,000/μL |
| | Neutrophil count ≤1,000/μL |
| | Pregnant or nursing (lactating) women |
| | Women of child-bearing potential, defined as all women |
| | physiologically capable of becoming pregnant, unless they use a |
| | highly effective method of contraception |
| | SjS: |
| | Sjögren's Syndrome overlap syndromes where another |
| | autoimmune disease constitutes the primary illness |
| | Required regular use of medications known to cause, as a major |
| | side effect, dry mouth/eyes. |
| Study Treatment | The compound of Formula (I), or a pharmaceutically acceptable salt |
| | thereof, capsules |
| | Placebo capsules |
| Efficacy | ESSDAI, ESSPRI, FACIT-F, PhGA, RCS, K-BILD, FVC, FEV, |
| assessments | salivary flow, Shirmer's test, DLCO |
| Pharmacodynamic | B cell activation (by monitoring of CD69 expression) after ex |
| assessments | vivo stimulation or non-stimulated whole blood. |
| | TNF secretion after ex vivo stimulation of whole blood |
| Pharmacokinetic | Serial blood samples for analysis of the compound of Formula |
| assessments | (I) concentrations and calculation of PK parameters (AUC, |
| | Cmax, Tmax and others) |
| Key safety | Adverse event monitoring |
| assessments | Physical examinations |
| | Vital signs |
| | Monitoring of laboratory markers in blood and urine |
| | Central ECG assessment |
| | Additional liver and renal safety monitoring |
| Data analysis | SjS |
| | A mixed effect model for repeated measurements (MMRM) will be |
| | fitted to the changes from baseline in ESSDAI for all time points |
| | until Week 24 with the treatment group, visit and treatment group by |
| | visit interaction and stratification factor screening ESSDAI score |
| | (<10 or ≥10) as fixed effects. An unstructured variance-covariance |
| | matrix will be fitted to model the dependency between repeated |
| | observations |
| | The difference between the compound of Formula (I) and placebo |
| | in change from baseline at each visit will be estimated from the |
| | model and presented with 95% confidence intervals. |
| | The following two criteria will be used to assess treatment efficacy |
| | a statistically significant reduction in ESSDAI at Week 24 in |
| | the compound of Formula (I) group compared to placebo, at |
| | one-sided 10% significance level, and |
| | an estimated mean reduction in ESSDAI at Week 24 in the |
| | compound of Formula (I) group to be at least 2 points |
| | greater than in placebo. |
| | A positive sign of efficacy will be considered if both criteria are met. |
| | MCTD |
| | A MMRM will be fitted to the changes from baseline in PhGA for all |
| | time points until Week 24 with the treatment group, visit and |
| | treatment group by visit interaction and stratification factor |
| | pulmonary involvement at baseline (yes/no) as fixed effects. A |
| | random intercepts model will be used. |
| | The difference between the compound of Formula (I) and placebo in |
| | change from baseline at each visit will be estimated from the model |
| | and presented with 95% confidence intervals. |

### Objectives and Endpoints

TABLE 1
Objectives and related endpoints

| Objective(s) | Endpoint(s) |
|---|---|
| Primary objective(s) | Endpoint(s) for primary objective(s) |
| SjS: To evaluate the efficacy of the compound of | SjS: Change from baseline in |
| Formula (I) compared to placebo based on change | ESSDAI at Week 24 |
| from baseline in ESSDAI at Week 24 | |
| MCTD: To evaluate the efficacy of the compound of | MCTD: Change from baseline in |
| Formula (I) compared to placebo based on change | PhGA at Week 24 |
| from baseline in Physician Global Assessment (PhGA) | |
| at Week 24 | |
| Secondary objective(s) | Endpoint(s) for secondary objective(s) |
| SjS/MCTD: To evaluate the efficacy of the compound | SjS: Change from baseline in |
| of Formula (I) compared to placebo based on change | ESSDAI, ESSPRI, FACIT-F and |
| from baseline on patient and physician-reported | PhGA over time up to Week 24 |
| outcomes over time up to Week 24 | MCTD: Change from baseline in |
| | FACIT-F, PhGA and ESSDAI |
| | (articular and pulmonary domains |
| | only) over time up to Week 24 |
| SjS/MCTD: To evaluate the safety and tolerability of | SjS/MCTD: Safety endpoints will |
| the compound of Formula (I) | include: |
| | Occurrence of treatment |
| | emergent adverse events (both |
| | serious and non-serious) during |
| | the study |
| | Occurrence of treatment |
| | emergent abnormal vital signs, |
| | laboratory and ECG values |
| | during the study |
| SjS/MCTD: To assess PK parameters of the | SjS/MCTD: PK parameters AUC, |
| compound of Formula (I) | Cmax, Tmax and others as needed at |
| | steady state |
| SjS: To explore the effect of the compound of Formula | SjS: Changes from baseline to the |
| (I) on quantitative salivary flow (unstimulated) over 24 | salivary flow rate over time up to 24 |
| weeks | weeks of treatment |
| SjS: To explore the effect of the compound of Formula | SjS: Changes from baseline to the |
| (I) on quantitative tear production over 24 weeks | Schirmer's test over time up to 24 |
| | weeks of treatment |
| MCTD: To evaluate the efficacy of the compound of | MCTD: Change from baseline in |
| Formula (I) based on change from baseline in Forced | FVC, FEV1, FEV2 and FEV3 over |
| Vital Capacity (FVC) and Forced Expiratory Volume | time up to Week 24 |
| (FEV1, FEV2, FEV3) over time up to Week 24 | |
| MCTD: To evaluate the efficacy of the compound of | MCTD: Change from baseline in the |
| Formula (I) based on change from baseline in the | diffusing capacity of lungs for carbon |
| diffusing capacity of lungs for carbon monoxide | monoxide (DLCO) over time up to |
| (DLCO) over time up to Week 24 | Week 24 |
| MCTD: To evaluate the efficacy of the compound of | MCTD: Change from baseline in |
| Formula (I) based on change from baseline in the | King's Brief Interstitial Lung Disease |
| patient reported outcome on lung function. | (K-BILD) over time up to week 24 |
| MCTD: To evaluate the efficacy of the compound of | MCTD: Change from baseline in RCS |
| Formula (I) based on change from baseline in | over time up to Week 24 |
| Raynaud's Condition Score (RCS) over time up to | |
| Week 24 | |

### Study Design

This is a phase 2 randomized, participant and investigator-blinded, placebo-controlled, multi-center, parallel group basket study to evaluate the safety, tolerability and efficacy of the compound of Formula (I) in patients with moderate to severe SjS or patients with diagnosis of MCTD. In case study participants receive concomitant therapy for their underlying disease and still meet entry criteria, they will remain on this therapy provided it remains stable until the end of the study.

A total of approximately 48 participants with SjS will be randomized in a 1:1 ratio to the compound of Formula (I) or placebo. A total of approximately 12 participants with MCTD will be randomized in a 1:1 ratio to the compound of Formula (I) or placebo.

Participants will first undergo a screening period of up to 6 weeks, followed by a treatment duration of 24 weeks and a follow-up period of 4 weeks. The total duration for each participant in the study will be up to 34 weeks.

Participants will receive 200 mg b.i.d. (200 mg twice per day) doses of the compound of Formula (I) or placebo.

Safety assessments will include physical examinations, ECGs, vital signs, standard clinical laboratory evaluations (hematology, biochemistry and urinalysis) as well as adverse event and serious adverse event monitoring.

### Screening

After signing informed consent, participants will be assessed for ESSDAI (SjS participants) and RCS and K-BILD (MCTD participants) as well as completing safety and other assessments to confirm RNP positivity and evaluate eligibility. For logistical reasons, assessments may be performed on different days, during the 6 week screening period, if deemed appropriate by the Investigator. Participants that fail screening, may be re-screened for one further occasion.

### Baseline

Eligible participants will return for the Baseline visit on Day 1. Participants may reside overnight at the site for logistical reasons, although this would not be considered a hospital admission. Eligibility must be confirmed prior to randomization and required baseline assessments must be completed prior to dosing on Day 1. If preferred by the site for scheduling purposes, some Baseline assessments may be carried out on the day prior to Day 1.

### Treatment

Treatment period lasts for 24 weeks, from week 0 (Day 1) to week 24 (Day 169). On Day 1, participants will be randomized to the respective SjS or MCTD treatment arms. Randomization of the SjS participants will be stratified at baseline by screening ESSDAI (< or ≥10 based on weighted scores) and randomization of the MCTD participants will be stratified by pulmonary involvement at baseline (yes/no).

Participants will return to the site at approximately 4-weekly intervals, with the exception of week 16 (Day 113) where only a safety phone call will be performed. As the compound of Formula (I) is administered for the first time in patients with SjS and MCTD, an additional visit will be performed at week 2 (Day 15) for safety and compliance purposes.

At the study visits, all participants will complete and/or undergo questionnaires (e.g. FACIT, PhGA), safety, PK, PD and biomarker sample collections.

The PK assessments will be performed pre dose and at various time points post dose (until 4 h after dosing) at week 4, week 12 and week 24. At week 4, an additional PK sample will be taken at 6 hours post dose, at selected sites.

Additionally, as per the Assessment Schedule, participant with SjS will:
undergo ESSDAI, salivary flow rate and Schirmer's test assessments
complete ESSPRI assessments.

Participants with MCTD will:
undergo ESSDAI (articular and pulmonary domains only), local spirometry, diffusing capacity of lungs for carbon monoxide (DLCO) test and nailfold capillaroscopy
complete Raynaud's Condition Score (RCS) and K-BILD assessments
perform exercise induced oxygen desaturation test

On the visit days, participants will be asked to take their morning dose of the study drug at the site. Participants will return their used drug supply packs for compliance and accountability assessment and will receive a new supply of study drug for the next 4 week treatment period (except at week 12 when the participants can chose to receive drug supply for 8 weeks treatment period).

Each week, participants will be asked to complete diaries to record administration of treatment.

All study visits will be ambulatory, however, for logistical reasons, it may be necessary for participants to come to the site the evening before their scheduled assessment visit. In these instances, the participants may stay overnight at the site, but this would not be considered a hospital admission.

Participants may be contacted by the Investigator/site staff during the study to ensure compliance/monitor safety by telephone or other means, if it is deemed appropriate or necessary by the Investigator.

The primary endpoint of the study will be assessed after the completion of 24-week treatment (Day 169; end of Week 24 visit).

### Follow Up and End of Study Visit (EoS)

After the last day of dosing, participants will enter a 4 week follow-up period without study drug treatment. Participants will be asked to return to the site at Week 28 for the End of Study visit. At this visit, participants will undergo final assessments.

Upon completion of this visit, participants will be discharged from the study.

### Rationale for Dose/Regimen and Duration of Treatment

The dose planned for this study is 200 mg b.i.d. (200 mg twice per day). This dose is expected to be safe and efficacious based on data from the first-in-human (FIH) clinical trial:
In the FIH study doses up to 1000 mg single administration and up to 400 mg b.i.d. (twice daily) for 14 days have been tested in healthy volunteers and proven to be safe and well-tolerated.
In the FIH study the dose of 200 mg b.i.d. (twice daily) for 14 days led to full pathway inhibition of both TLR7 and TLR8, and is therefore expected to provide maximum clinical efficacy.

The study treatment duration is 24 weeks to allow for a meaningful assessment of the safety, tolerability and efficacy of the compound of Formula (I) in SjS and MCTD. The 24-week treatment period is based on recent internal clinical trial data suggesting that meaningful treatment difference versus placebo, in terms of clinical efficacy, can be expected by week 24, and a pharmacological efficacy plateau could be reached within 12 to 24 weeks of treatment.

Based on analysis of PK and PD data from the first-in-human study, the compound of Formula (I) can be expected to have fast onset of pharmacological action, however, time to a meaningful improvement in clinical efficacy parameters, such as ESSDAI or ESSPRI, might take up to 24 weeks.

A follow-up period of 4 weeks following the last dose is will be implemented for adverse event monitoring. No pharmacological effect is expected beyond 3 days (=5 times terminal half-life) after the end of treatment, based on the short terminal half-life of the compound of Formula (I) of max. 14.4 hours.

### Rationale for Choice of Control Drug (Placebo)

The comparator treatment in the study will be placebo, in order to provide objective control for the evaluation of safety, clinical efficacy and PD during the 24-week treatment with the compound of Formula (I).

Since there is no approved systemic treatment for SjS and MCTD, the use of placebo as comparator is considered justified. Current standard of care for SjS and MCTD patients is often limited to symptomatic care of symptoms (e.g. dryness or Raynaud's phenomenon). Steroids and conventional DMARDs are often ineffective. No pharmacologic intervention is effective against the severe, disabling fatigue associated with SjS.

What is claimed is:

1. A method of treating Sjögren's Syndrome in a subject in need thereof, comprising administering, e.g. orally, to the subject a compound of Formula (I), or a pharmaceutically acceptable salt thereof, 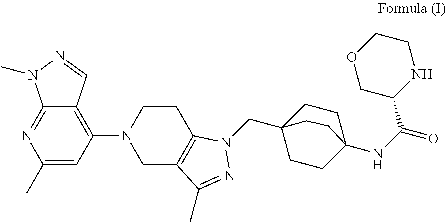 [SMILES: Cc1cc(N2CCc3c(c(C)nn3CC34CCC(NC(=O)[C@@H]5COCCN5)(CC3)CC4)C2)c2cnn(C)c2n1]
at a dose of about 25 mg to about 400 mg, e.g. about 50 mg to about 300 mg, e.g. about 100 mg to about 200 mg, twice daily, e.g. about every 12 hours.

2. The method of claim 1, wherein the dose is about 100 mg to about 200 mg, twice daily, e.g. about every 12 hours.

3. The method of claim 1, wherein the dose is about 200 mg twice daily, e.g. about every 12 hours.

4. The method of claim 1, wherein the subject has moderate to severe Sjögren's Syndrome.

5. The method of claim 1, wherein the subject e.g., a patient, is positive, e.g. seropositive, for anti-extractable nuclear (ENA) antibodies, e.g. anti-Sjögren's syndrome related antigen A (anti-Ro/SSA) antibodies.

6. A compound of Formula (I): 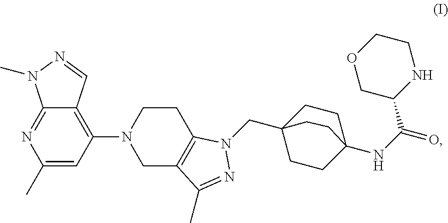 [SMILES: Cc1cc(N2CCc3c(c(C)nn3CC34CCC(NC(=O)[C@@H]5COCCN5)(CC3)CC4)C2)c2cnn(C)c2n1]
or a pharmaceutically acceptable salt thereof, for use in treating Sjögren's syndrome (SjS) in a subject in need thereof, wherein the compound of Formula (I), or a pharmaceutically acceptable salt thereof is administered at a dose of about 25 mg to about 400 mg, e.g. about 50 mg to about 300 mg, e.g. about 100 mg to about 200 mg, twice daily, e.g. about every 12 hours.

7. A method of treating mixed connective tissue disease in a subject in need thereof, comprising administering, e.g. orally, to the subject a compound of Formula (I), or a pharmaceutically acceptable salt thereof, 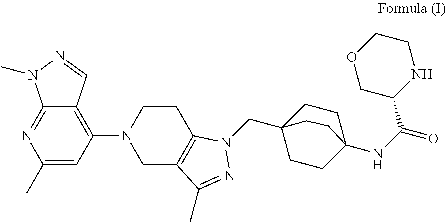 [SMILES: Cc1cc(N2CCc3c(c(C)nn3CC34CCC(NC(=O)[C@@H]5COCCN5)(CC3)CC4)C2)c2cnn(C)c2n1]
at a dose of about 25 mg to about 400 mg, e.g. about 50 mg to about 300 mg, e.g. about 100 mg to about 200 mg, twice daily, e.g. about every 12 hours.

8. The method of claim 7, wherein the dose is about 100 mg to about 200 mg, twice daily, e.g. about every 12 hours.

9. The method of claim 7, wherein the dose is about 200 mg twice daily, e.g. about every 12 hours.

10. The method of claim 1, wherein the subject, e.g., a patient, is positive, e.g. seropositive, for anti-extractable nuclear (ENA) antibodies, e.g. anti-U1-RNP antibodies.

11. A compound of Formula (I): 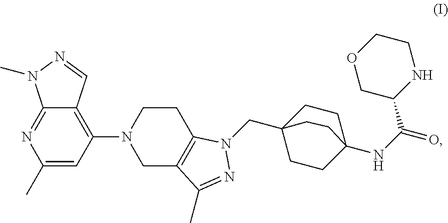 [SMILES: Cc1cc(N2CCc3c(c(C)nn3CC34CCC(NC(=O)[C@@H]5COCCN5)(CC3)CC4)C2)c2cnn(C)c2n1]
or a pharmaceutically acceptable salt thereof, for use in treating mixed connective tissue disease in a subject in need thereof, wherein the compound of Formula (I), or a pharmaceutically acceptable salt thereof is administered at a dose of about 25 mg to about 400 mg, e.g. about 50 mg to about 300 mg, e.g. about 100 mg to about 200 mg, twice daily, e.g. about every 12 hours.
